(19) United States
(12) Patent Application Publication
(10) Pub. No.: US 2024/0339047 A1
(43) Pub. Date: Oct. 10, 2024

(54) DENTAL IMAGING TEACHING APPARATUS

(71) Applicant: Fangkai HAN, Shanghai (CN)

(72) Inventors: Fangkai HAN, Shanghai (CN); Jue LI, Shanghai (CN); Zekuan YU, Shanghai (CN)

(73) Assignee: Fangkai HAN, Shanghai (CN)

(21) Appl. No.: 18/700,008

(22) PCT Filed: Oct. 15, 2021
(86) PCT No.: PCT/CN2021/124051

§ 371 Date: Apr. 10, 2024

(51) Int. Cl. G09B 23/28; A61B 1/24

(52) U.S. Cl. CPC G09B 23/286; A61B 1/24

(57) ABSTRACT

A dental imaging teaching apparatus includes a light source, a tooth model and an imaging plate. The light source is configured to emit visible light, and the tooth model is at least partially transparent to visible light. The imaging plate is provided with graduations in the form of a grid. The tooth model includes a plurality of nodes and connections. The plurality of nodes is connected by the connections, forming a mesh. This dental imaging teaching apparatus avoids the ambiguities and operating difficulties associated with the conventional X-ray projection techniques, enhances radiography technology and reduces imaging repetitions, effectively reducing a patient's unnecessary exposure to the harmful radiation due to such repetitions. Moreover, it can be easily applied to teaching and professional education, and as the first periapical projection teaching apparatus, it improves the theoretical system of periapical projection technology for oral and maxillofacial imaging for medical use.

### FIELD OF THE INVENTION

Embodiments herein relate to the field of oral and maxillofacial imaging for medical use and, in particular, to a dental imaging teaching apparatus.

### DESCRIPTION OF THE PRIOR ART

In the oral clinical practice, intraoral periapical (dental) radiography refers to capture of a 2D image of a tooth exposed in an x-ray beam projected from a dental X-ray machine on an image receptor (film or imaging plate). Projection techniques used in dental X-ray periapical radiography include the two described below.

1) Bisecting-angle projection is the most commonly used projection technique in the current clinical practice, in which a central X-ray is configured perpendicular to an angle bisector between a long axis of a tooth being treated and an image receptor. The WS/T 608-2018 standard of the People's Republic of China (PRC) for the health industry, titled “Basic Operation of Oral and Maxillofacial Conventional X-Ray Practices, (referred to hereinafter as the “Standard”) specifies that “a central X-ray should be perpendicular to an angle bisector between a long axis of a tooth being treated and an image receptor, and should be parallel to a tangent of a proximal surface of the tooth as much as possible”.

In the bisecting-angle projection technique, an X-ray beam is projected perpendicular to an angle bisector between a tooth and an image receptor, producing an image of the tooth onto the image receptor. Theoretically, the tooth image on the image receptor has the same length as the true length of the tooth because of the angle bisector. The section “Placement and Securing of Image Receptor” of the Standard recites that “the image receptor should be placed in the mouth so that a photosensitive surface of the image receptor is brought into close contact with the lingual (palatal) surface”. However, the Standard does not clearly define a spatial position for the tooth's long axis. As both the image receptor and the tooth are located in the mouth, the angle between them cannot be identified by naked eye, making the method itself ambiguous. No parameters are clearly defined for the measurement of the angle. The Standard requires that “a central X-ray projected for periapical radiography of a tooth being treated should pass through the middle of the root of the tooth”. Here, the definition of the “middle” remains vague. As for how to determine a projection direction in this technique, the Standard recommends using body surface features as fiducial markers. Specifically, a) for a maxillary tooth, with a line connecting the upper edge of the external auditory meatus and the tip of the nose being taken as an imaginary line, the central X-ray may pass 1) the tip of the nose in case of a maxillary central incisor; 2) a midpoint of a line segment connecting the tip of the nose and the ala of the nose on the same side in case of a maxillary central or lateral incisor on the side; or 3) the ala of the nose on the same side in case of a maxillary cuspid; and b) for any mandibular tooth, the imaginary line may run 10 mm above the lower edge of the mandible, and the central X-ray may be projected in alignment with the tooth. As can be seen, although the object to be imaged is a tooth, which is intraoral hard tissue, none of the projection direction and angle and reference points that are used in this technique to locate a tooth is directly related to the tooth. Instead, a spatial position of the tooth is estimated or predicted based on aligning the bulb tube with a facial soft tissue landmark, imaginary line, plane or the like. Therefore, it fails to allow for accurate projection in a desired direction or at a desired angle in actual clinical practice.

This projection technique does not take into account various factors such as different projection angles, different projection directions, and even different operating dentists, tooth morphology variation among patients and spatial position variation. Consequently, the same tooth may be imaged with different morphologies at a given projection angle using the projection technique due to uncertainties thereof in projection angle. Moreover, due to individual tooth variation, there may be significant differences in spatial position in estimates made based on a given body surface landmark. Thus, this technique would undoubtedly introduce great errors and uncertainties to clinically obtained images.

2) Periapical paralleling is a projection technique seldom used in the clinical practice, in which X-rays are horizontally projected onto a tooth being treated from an X-ray generator (bulb tube) which is position as far as possible from the tooth. The projection features use of a long beam-limiting cone.

The periapical paralleling technique, also known as the right-angle technique, long-beam-limiting cone technique, or long focal length paralleling technique, operates by placing an image receptor in parallel to a long axis of a tooth and projecting a central X-ray perpendicular both to the long axis of the tooth and to the image receptor. Theoretically, images taken using this technique have least distortion. In order to ensure parallelism of the film to the long axis of the tooth, the film has to be placed away from the tooth. Moreover, a high voltage and a fast film are used to reduce the time and amount of exposure. However, this method requires the use of necessary supporting tools, considerable time consumption and occupation of a relatively large intra-oral space for the placement of the film. As seen in the clinical practice, in most cases, the parallelism of a film to a long axis of a tooth cannot be ensured within the oral cavity due to an inclination of the teeth, the morphology, thickness and other factors of the surrounding soft tissues (the tongue, the floor of the mouth and the palate) and bones (the alveolar bone, the palatal arch), the volume of the oral cavity and difficulties in patient cooperation.

As can be seen from the above description of the Standard for the two techniques, in the current practice of intraoral periapical radiography, despite the guidelines provided in the Standard, for dentists, there is still a lack of reliable guidance on their practical operation. Therefore, they have to resort to accumulating experience in practical operation to improve their skills. Although teaching apparatuses are available for practicing, the following problems remain:
1. The intraoral periapical radiography teaching apparatuses use X-ray sources, which are expensive and require the use of a variety of protective equipment for ensuring radiation safety. Thus, these apparatuses are complicated and associated with demanding environmental requirements.
2. X-rays are invisible and do not produce a visible spot which can serve as a straightforward locating aid during operation, and the exposed film has to be relied on, which is, however, an indirect means for locating. Therefore, using X-rays in teaching is inefficient.
3. Common tooth models (or specimens) are opaque, making it impractical to use a visible light source. Visible light can be only used to visualize the contour of a tooth on a film and cannot be used to demonstrate the internal structure thereof.
4. Films used in the current clinical practice do not allow direct marked of the size and position of a tooth image formed thereon and requires identification with separate software, making the teaching apparatuses inefficient. In addition, such films are consumables, which cannot be used any longer once exposed. This will lead to both a waste and a decrease in teaching efficiency.

Therefore, those skilled in the art are directing their efforts toward developing a dental imaging teaching apparatus, which can overcome the above-described problems with the prior art.

### SUMMARY OF THE INVENTION

In view of the above-described drawbacks of the prior art, the present invention provides a dental imaging teaching apparatus comprising a tooth model, which is at least partially transparent to visible light.

Preferably, the tooth model comprises a plurality of nodes and connections, which connect the plurality of nodes, thus forming the tooth model in the form of a mesh.

Preferably, the connections are configured to be deformable to change relative distances and positions of the plurality of nodes.

Preferably, the tooth model comprises a first set of nodes, which has a cross-section of a first shape and is configured to indicate a mesial area of the tooth model.

Preferably, the tooth model comprises a second set of nodes, which has a cross-section of a second shape and is configured to indicate a middle area of the tooth model.

Preferably, the tooth model comprises a third set of nodes, which has a cross-section of a third shape and is configured to indicate a distal area of the tooth model.

Preferably, the tooth model comprises a fourth set of nodes, which has a cross-section of a fourth shape and is configured to indicate a pulp of the tooth model.

Preferably, the tooth model comprises a fifth set of nodes, which has a cross-section of a fifth shape and is configured to indicate a coronal plane of the tooth model.

Preferably, the tooth model comprises a sixth set of nodes, which has a cross-section of a sixth shape and is configured to indicate a transverse plane of the tooth model.

Preferably, the tooth model comprises a seventh set of nodes, which has a cross-section of a seventh shape and is configured to indicate a sagittal plane of the tooth model.

Preferably, in the tooth model, the other space than a space occupied by the nodes and the connections is filled with a material at least partially transparent to visible light.

Preferably, in the tooth model, the nodes and the connections are made of materials at least partially transparent to visible light.

Preferably, the dental imaging teaching apparatus further comprises a light source comprising a light source body, a light source support and a light source base. The light source body is connected to the light source base by the light source support.

Preferably, the light source body is configured to emit visible light.

Preferably, a first movable device is provided at a junction of the light source support and the light source base. The first movable device is used to adjust the light source in height and angle.

Preferably, the tooth model comprises a tooth model body, a tooth model support and a tooth model base. The tooth model body is connected to the tooth model base by the tooth model support.

Preferably, a second movable device is provided at a junction of the tooth model support and the tooth model base. The second movable device is used to adjust the tooth model in height and angle.

Preferably, the dental imaging teaching apparatus further comprises an imaging plate comprising an imaging plate body, an imaging plate support and an imaging plate base. The imaging plate body is connected to the imaging plate base by the imaging plate support.

Preferably, a third movable device is provided at a junction of the imaging plate support and the imaging plate base. The third movable device is used to adjust the imaging plate in height and angle.

Preferably, the imaging plate body is provided with graduations in the form of a grid.

Compared with the prior art, the present application has at least the effects as follows:
1) The relationship of an X-ray tube, a tooth and an imaging plate is visualized using visible light according to the same image principles, avoiding ionizing radiation hazards that may arise from the use of X-rays.
2) Imaging can be conducted in a continuous and immediate manner, compared with the conventional X-ray imaging which must be performed in an intermittent fashion with delays because each image is obtained only after exposure. In contrast, using visible light enables immediate imaging that features more intuitive demonstration of continuous variation of a relationship of light, an object and an image.
3) The tooth model is innovatively designed with marking nodes arranged into a matrix or mesh. Each node represents a different tooth surface or internal structure, and the nodes are connected by continuous connections. The marking nodes may have different geometric properties such as shape and thickness and/or different colors to comprehensively and intuitively demonstrate relationships among the nodes in the 3D space. The marking nodes may be equidistantly arranged to facilitate identification of deformation patterns.
4) Details can be presented in real time. Projections of marking nodes spaced from the light source at different distances move at the same time in distinct patterns as the light source is moving (oblique projection). Projections of nodes on the side closer to the light source move greater distances than those of nodes on the other side. These variation patterns can be demonstrated on the graduated imaging plate with concrete values.

Positional relationships between teeth at different positions in space and between adjacent teeth, such as those with non-parallel axes, can be simulated, such as a labial-palatal relationship of adjacent teeth. Variation of a labial-palatal relationship of nodes within a single tooth, the spatial configuration of a tooth root, variation of different tooth roots, etc. can be simulated. Deformation regularities can be accurately reflected with a graduated scale on the imaging plate. Positional variation of nodes in the same tooth or of different teeth can be comprehensively presented in real time. Thus, imaging regularities can be demonstrated through real-time light and image variation.
5) The deformability of the tooth model enables simulation of various numbers of tooth roots of different shapes.
6) Variables can be adjusted flexibly. One, two or three variables may be derived from relationship among the aforementioned three components. With the aid of feedback of given nodes in an image, positional relationships among them can be perfectly presented in the image.
7) The essence of intraoral periapical projection simulation enabled by the dental imaging teaching apparatus is a positional relationship of given nodes in space. Firstly, it avoids the ambiguities and operating difficulties associated with the conventional X-ray projection techniques. Secondly, it enhances radiography technology and reduces imaging repetitions, effectively reducing a patient's unnecessary exposure to the harmful radiation due to such repetitions. Thirdly, it can be easily applied to teaching and professional education. Fourthly, as the first periapical projection teaching apparatus, it improves the theoretical system of periapical projection technology for oral and maxillofacial imaging for medical use.

Below, the concept, structural details and resulting technical effects of this application will be further described with reference to the accompanying drawings to provide a full understanding of the objects, features and effects of the application.

### BRIEF DESCRIPTION OF THE DRAWINGS

FIG. 1 schematically illustrates the overall structure of an embodiment of the present application.

FIG. 2 is a top view of an arrangement of nodes in a tooth model according to an embodiment of the present application.

FIG. 3 is a side view of an arrangement of nodes in a tooth model according to an embodiment of the present application.

FIG. 4 is a side view of an arrangement of nodes in a tooth model according to an embodiment of the present application.

FIG. 5 is a side view of an arrangement of nodes in a tooth model according to an embodiment of the present application.

FIG. 6 is a side view of an arrangement of nodes in a tooth model according to an embodiment of the present application.

FIG. 7 is a side view of an arrangement of nodes in a tooth model according to an embodiment of the present application.

FIG. 8 is a side view of an arrangement of nodes in a tooth model according to an embodiment of the present application.

FIG. 9 is a top view of an arrangement of nodes in a tooth model according to an embodiment of the present application.

FIG. 10 is a side view of an arrangement of nodes in a tooth model according to an embodiment of the present application.

FIG. 11 is a side view of an arrangement of nodes in a tooth model according to an embodiment of the present application.

FIG. 12 is a side view of an arrangement of nodes in a tooth model according to an embodiment of the present application.

FIG. 13 is a top view of an arrangement of nodes in a tooth model according to an embodiment of the present application.

FIG. 14 is a side view of an arrangement of nodes in a tooth model according to an embodiment of the present application.

FIG. 15 is a side view of an arrangement of nodes in a tooth model according to an embodiment of the present application.

FIG. 16 is a top view of an arrangement of nodes in a tooth model according to an embodiment of the present application.

FIG. 17 is a schematic diagram showing the structure of a light source according to an embodiment of the present application.

FIG. 18 is a schematic diagram showing the structure of a tooth model according to an embodiment of the present application.

FIG. 19 is a schematic diagram showing the structure of an imaging plate according to an embodiment of the present application.

FIG. 20 schematically illustrates images taken at different angles of a light source according to an embodiment of the present application.

FIG. 21 schematically illustrates images taken at different angles of a tooth model according to an embodiment of the present application.

FIG. 22 schematically illustrates images taken at different angles of an imaging plate according to an embodiment of the present application.

### DETAILED DESCRIPTION OF THE PREFERRED EMBODIMENTS

A few preferred embodiments of the present invention will be described more fully hereinafter with reference to the accompanying drawings so that techniques thereof will become more apparent and more readily understood. The invention can be embodied in various different forms and its scope is in no way limited to the embodiments discussed herein.

Throughout the figures, structurally identical components are indicated with the same reference numerals, and structurally or functionally similar components are indicated with like reference numerals. The dimensions and thickness of each component in the accompanying drawings are arbitrarily shown, and the present invention is not limited to any particular dimension or thickness of any component. In the figures, where appropriate, the thickness of certain components may be somewhat exaggerated for clarity. As used herein, the term “cross-sectional shape” refers to the shape of an image formed on an imaging plate by projected light.

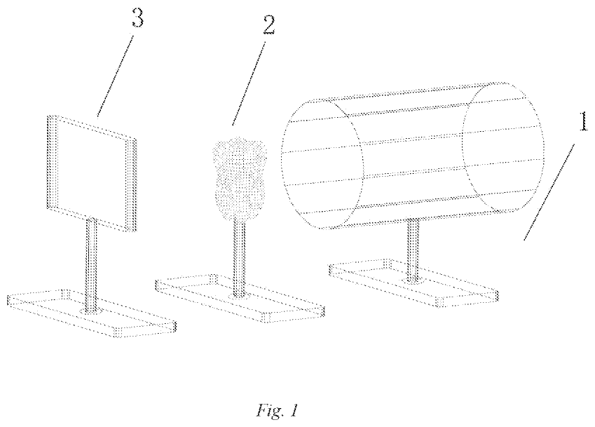

Embodiments of the present invention provide a dental imaging teaching apparatus as shown in FIG. 1, which includes a light source 1, a tooth model 2 and an imaging plate 3. The light source 1 projects visible light onto the tooth model 2 to form an image thereof on the imaging plate 3.

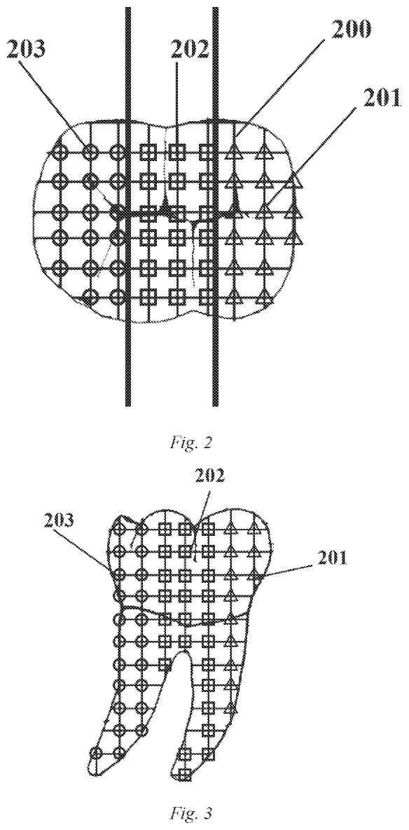

Preferably, the tooth model 2 is structured as particularly shown in FIGS. 2 to 5. The tooth model 2 is composed of a plurality of nodes and connections. As shown in FIG. 2, in this embodiment, the connections are implemented as thin struts 200, which connect adjacent pairs of the nodes 21, 22, 23, thus forming the tooth model 2 in the form of a mesh. The rest of the tooth model 2, except for the nodes and connections, is transparent to visible light. That is, visible light can at least partially pass through the tooth model 2. The rest of the tooth model 2, except for the nodes and connections, may be hollow, in order to allow the tooth model 2 to have a reduced weight and lower cost. Alternatively, it may be made of a material partially transparent to visible light, such as glass or a plastic material, in order to allow the tooth model 2 to have increased strength. In some preferred embodiments, the thin struts 200 serving as the connections are telescopic and thus variable in length, allowing adjustable distances and relative positions of the nodes. As a result of visible light being projected from the light source 1 onto the tooth model 2, an image of the imaging plate 3 will be formed also in the form of a mesh.

As shown in FIG. 2, in this embodiment, the nodes in the tooth model 2 include a first set of nodes 201, a second set of nodes 202 and a third set of nodes 203. In the figure, the second set of nodes 202 is represented with squares and used to indicate a middle area of the tooth model 2. That is, the second set of nodes 202 is arranged in the middle area of the tooth model 2. In the figure, the first set of nodes 201 is represented with triangles and used to indicate a mesial area of the tooth model 2. That is, the first set of nodes 201 is arranged in the mesial area of the tooth model 2. In the figure, the third set of nodes 203 is represented with circles and used to indicate a distal area of the tooth model 2. That is, the third set of nodes 203 is arranged in the distal area of the tooth model 2. It should be noted that, as used herein, the terms “distal” and “mesial” are merely intended to distinguish between different areas, rather than imply any particular positional relationship. In practice, the first set of nodes 201 may be each made with a triangular cross-section, the second set of nodes 202 with a square cross-section, and the third set of nodes 203 with a circular cross-section, as shown in FIGS. 2 to 5. Other cross-sectional shapes are possible, as long as the different sets of nodes can be distinguished from one another. Since projection imaging is a process to form an image of a three-dimensional (3D) object on a two-dimensional (2D) plane, and some 3D information of the object, such as its thickness, may be lost from the 2D image obtained by the process. Typically, for some features of a tooth that are visible on a 2D image thereof, it is not possible to determine whether they are located in a mesial, middle or distal area of the tooth. In contrast, according to this embodiment, through arranging nodes with different cross-sectional shapes in the mesial, middle and distal areas of the tooth model 2, more 3D information can be obtained from a 2D image of the object. For example, it is possible to determine whether a portion of the image is formed as a result of projection of node(s) in a single one of the mesial, middle and distal areas, or superimposed projection of nodes in two or three of the areas.

FIGS. 3 to 5 schematically illustrate the structure of the tooth model 2 according to this embodiment, observed at different angles. As shown in FIG. 3, when observing the tooth model 2 from one side, the observer can see all of the first set of nodes 201 in the mesial area, the second set of nodes 202 in the middle area and the third set of nodes 203 in the distal area. In an image formed by projecting light onto the tooth model 2 from this perspective, the first set of nodes 201 will be located on the right of the image, the second set of nodes 202 in the middle of the image, and the third set of nodes 203 on the left of the image.

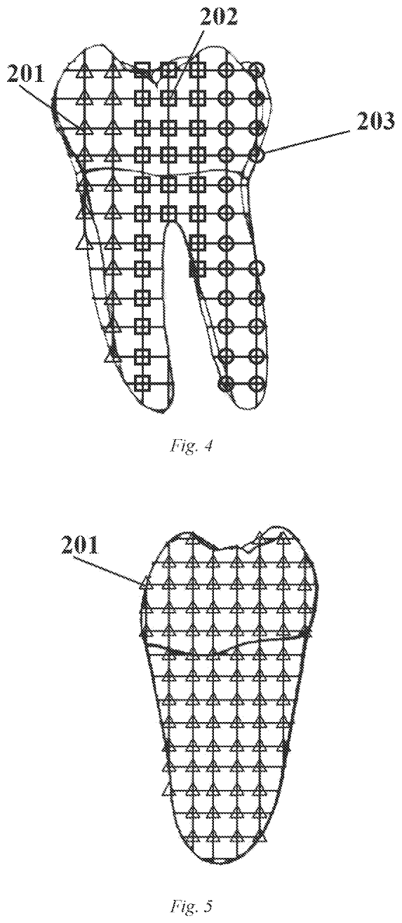

As shown in FIG. 4, when observing the tooth model 2 from another side, the observer can also see all of the first set of nodes 201 in the mesial area, the second set of nodes 202 in the middle area and the third set of nodes 203 in the distal area. In an image formed by projecting light onto the tooth model 2 from this perspective, the first set of nodes 201 will be located on the left of the image, the second set of nodes 202 in the middle of the image, and the third set of nodes 203 on the right of the image.

As shown in FIG. 5, when observing the tooth model 2 from yet another side, the observer can see only the first set of nodes 201 in the mesial area but not the second set of nodes 202 in the middle area and the third set of nodes 203 in the distal area due to occlusion by the first set of nodes 201. In an image formed by projecting light onto the tooth model 2 from this perspective, the first set of nodes 201, the second set of nodes 202 and the third set of nodes 203 will be superimposed with one another.

In some embodiments, a fourth set of nodes is further included, which is preferred to indicate the pulp. Notably, the pulp is three-dimensionally (3D) present in the tooth and occupies its entire internal space. Accordingly, some of the fourth set of nodes that indicates the pulp are arranged in the middle area, and the remaining ones may be arranged in one or both of the mesial and distal areas. Moreover, the fourth set of nodes is configured with a cross-sectional shape differing from those of the aforementioned three sets of nodes. Preferably, the cross-sectional shape of the fourth set of nodes is pentagonal. As nodes in the fourth set in different areas are adjacent to differently shaped nodes, the area in which a certain node in the fourth set is present can be determined from the shape of adjacent nodes.

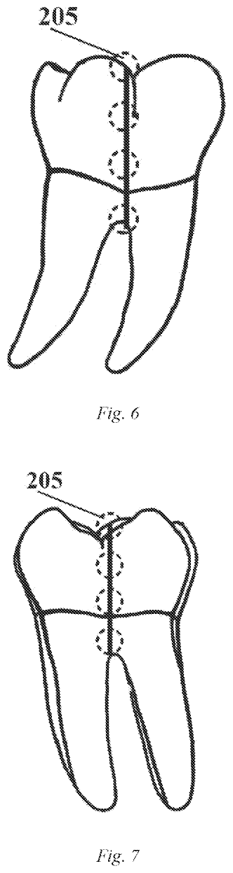

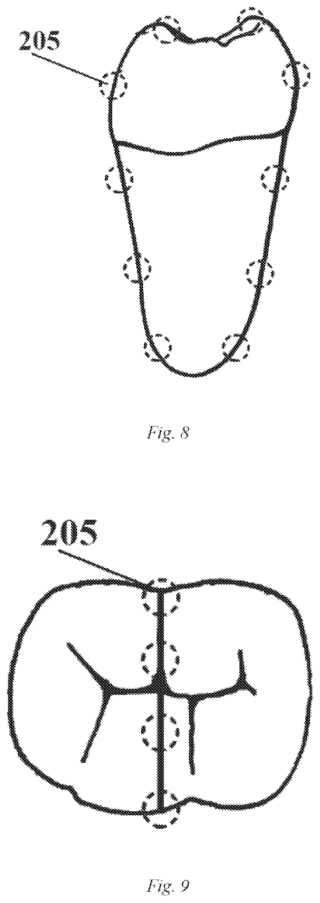

As shown in FIGS. 6 to 9, in this embodiment, the tooth model 2 further includes a fifth set of nodes 205, which indicates the coronal plane of the tooth. In FIGS. 6 to 9, in order to be distinguished from the nodes in the other sets, the fifth set of nodes 205 is represented by dashed circles. In practical fabrication, it is unnecessary for these nodes to be made with the shown shape, as long as their cross-sectional shape is different from those of the other nodes. The fifth set of nodes 205 is scattered across the coronal plane of the tooth model 2. When the tooth model 2 is observed at some angles, the fifth set of nodes 205 appears to be aligned almost in a straight line, as shown in FIGS. 6, 7 and 9. When the tooth model 2 is observed at another angle, the fifth set of nodes 205 appears to be located on the outer contour, as shown in FIG. 8.

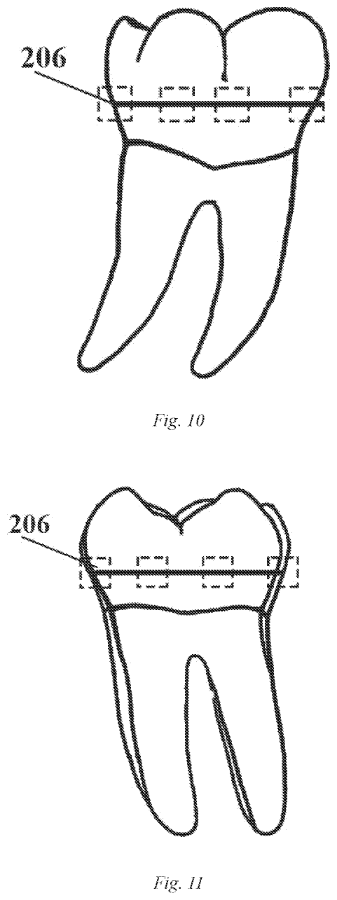

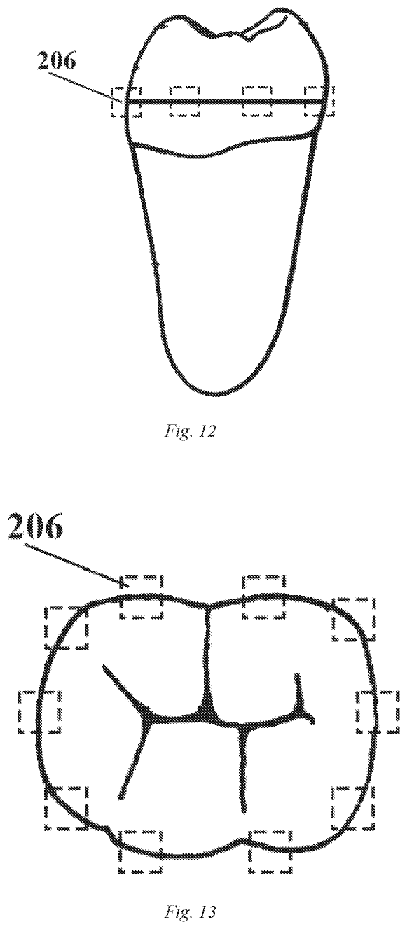

As shown in FIGS. 10 to 13, in this embodiment, the tooth model 2 further includes a sixth set of nodes 206, which indicates a transverse plane of the tooth. In FIGS. 10 to 13, in order to be distinguished from the nodes in the other sets, the sixth set of nodes 206 is represented by dashed squares. In practical fabrication, it is unnecessary for these nodes to be made with the shown shape, as long as their cross-sectional shape is different from those of the other nodes. The sixth set of nodes 206 is arranged in the transverse plane of the tooth model 2. When the tooth model 2 is observed at some angles, the sixth set of nodes 206 appears to be aligned almost in a straight line, as shown in FIGS. 10 to 12. When the tooth model 2 is observed at another angle, the sixth set of nodes 206 appears to be located on the outer contour, as shown in FIG. 13.

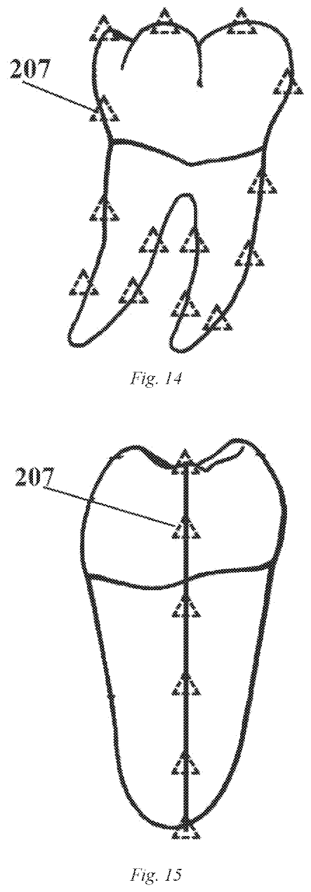

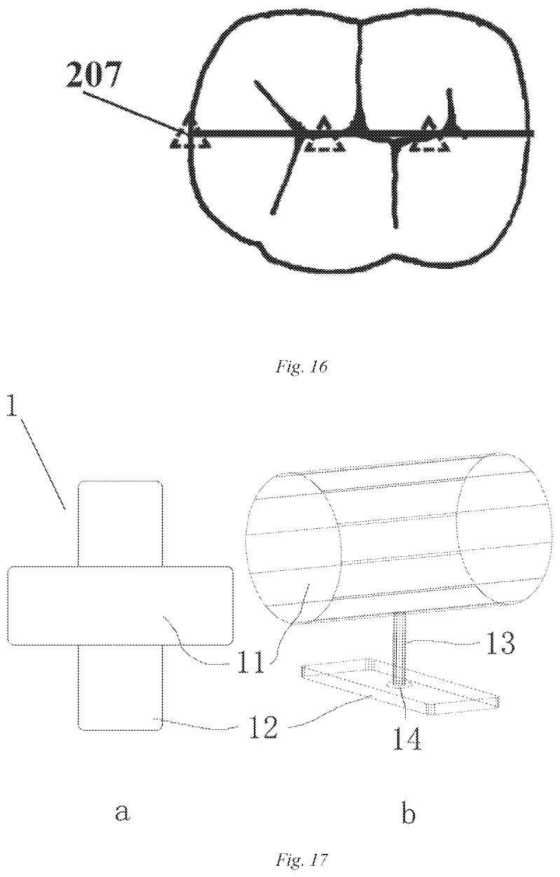

As shown in FIGS. 14 to 16, in this embodiment, the tooth model 2 further includes a seventh set of nodes 207, which indicates a sagittal plane of the tooth. In FIGS. 14 to 16, in order to be distinguished from the nodes in the other sets, the seventh set of nodes 207 is represented by dashed triangles. In practical fabrication, it is unnecessary for these nodes to be made with the shown shape, as long as their cross-sectional shape is different from those of the other nodes. The seventh set of nodes 207 is arranged in the sagittal plane of the tooth model 2. When the tooth model 2 is observed at some angles, the seventh set of nodes 207 appears to be aligned almost in a straight line, as shown in FIGS. 15 to 16. When the tooth model 2 is observed at another angle, the seventh set of nodes 207 appears to be located on the outer contour, as shown in FIG. 17.

In some embodiments, the nodes and connections are made of a material partially transparent to visible light, such as colored glass, plastic, quartz, etc. For example, the first set of nodes 201 that indicates the mesial area may be made of a yellow transparent material. The second set of nodes 202 that indicates the middle area may be made of a grey transparent material. The third set of nodes 203 that indicates the distal area may be made of a purple transparent material. The fourth set of nodes that indicates the pulp may be made of a red transparent material. The fifth set of nodes 205 that indicates the coronal plane may be made of a blue transparent material. The connections that connect the nodes may also be made of materials with the respective colors, for the purpose of distinguishing from one another. In some embodiments, the connections may be made with different thicknesses, for the purpose of distinguishing from one another. In some embodiments, a plurality of tooth models 2 may be provided, which represent different types of teeth, in order to simulate a real intraoral situation.

FIG. 17 shows a schematic diagram illustrating the structure of the light source 1 in this embodiment. The light source 1 includes a light source body 11, a light source base 12 and a light source support 13. The light source body 11 is connected to the light source base 12 by the light source support 13. In FIG. 17, Detail a shows a top view of the light source 1, and Detail b shows a perspective view of the light source 1. In this embodiment, the light source body 11 is cylindrical in shape. As can be seen from Detail a of FIG. 17, the cylindrical light source body 11 is arranged substantially perpendicular to the light source base 12. The cylindrical light source body 11 can collimate visible light emitted from a light-emitting element, allowing the light source 1 to produce parallel visible light. A first movable device 14 is provided at the junction of the light source support 13 and the light source base 12. The light source support 13 is rotatably connected to the light source base 12 by the first movable device 14, allowing parallel visible light to be emitted from the light source 1 at an adjustable height and angle.

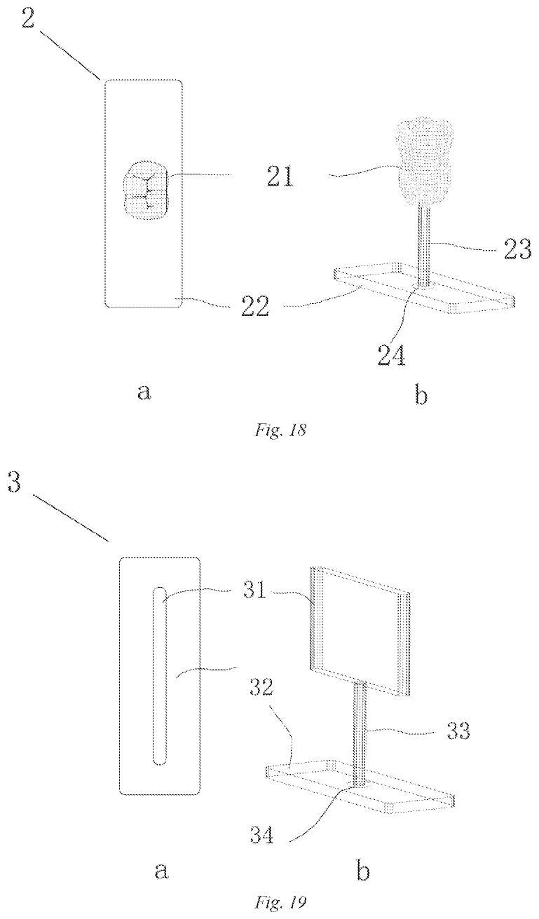

FIG. 18 shows a schematic diagram illustrating the structure of the tooth model 2 in this embodiment. The tooth model 2 includes a tooth model body 21, a tooth model base 22 and a tooth model support 23. The tooth model body 21 is connected to the tooth model base 22 by the tooth model support 23. In FIG. 18, Detail a shows a top view of the tooth model 2, and Detail b shows a perspective view of the tooth model 2. The tooth model body 21 is in the form of a mesh and is at least partially transparent to visible light. A second movable device 24 is provided at the junction of the tooth model support 23 and the tooth model base 22. The tooth model support 23 is rotatably connected to the tooth model base 22 by second movable device 24, allowing the tooth model body 21 to be adjusted in height, angle and orientation.

FIG. 19 is a schematic diagram showing the structure of the imaging plate 3 in this embodiment. The imaging plate 3 includes an imaging plate body 31, an imaging plate base 32 and an imaging plate support 33. The imaging plate body 31 is connected to the imaging plate base 32 by the imaging plate support 33. In FIG. 19, Detail a shows a top view of the imaging plate 3, and Detail b shows a perspective view of the imaging plate 3. The imaging plate body 31 is in the shape of a parallel plate and is disposed substantially parallel to the imaging plate base 32. A third movable device 34 is provided at the junction of the imaging plate support 33 and the imaging plate base 32. The imaging plate support 33 is rotatably connected to the imaging plate base 32 by the third movable device 34, allowing the imaging plate 31 to be adjusted in height and angle. Preferably, the imaging plate body 31 is marked with graduations in the form of a grid.

The teaching apparatus of the present application allows a user not only to be trained on correct dental imaging, but also to accumulate valuable experience from image distortions resulting from positional deviations of the light source, tooth or imaging plate that may occur during practicing attempts. Moreover, in this embodiment, the first movable device 14, the second movable device 24, the third movable device 34 allow the light source 1, the tooth model 2 and the imaging plate 3, respectively, to be separately adjusted in height and angle. This entails a parametric control approach, in which the position or angle of only one of the light source 1, the tooth model 2 and the imaging plate 3 can be adjusted each time, allowing a user to accumulate experience in corresponding image distortion patterns.

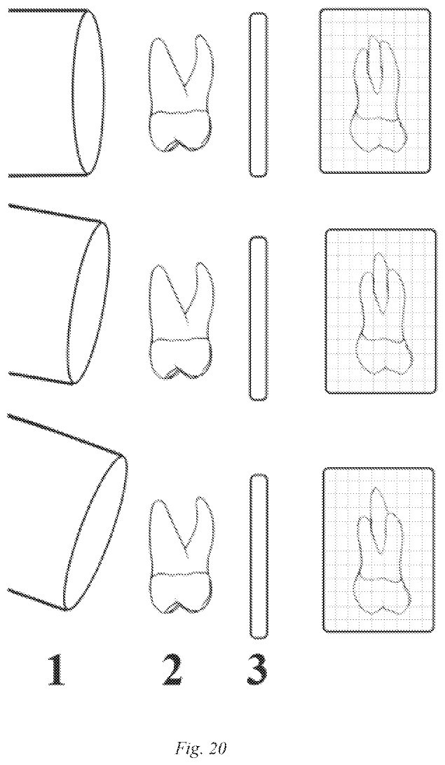

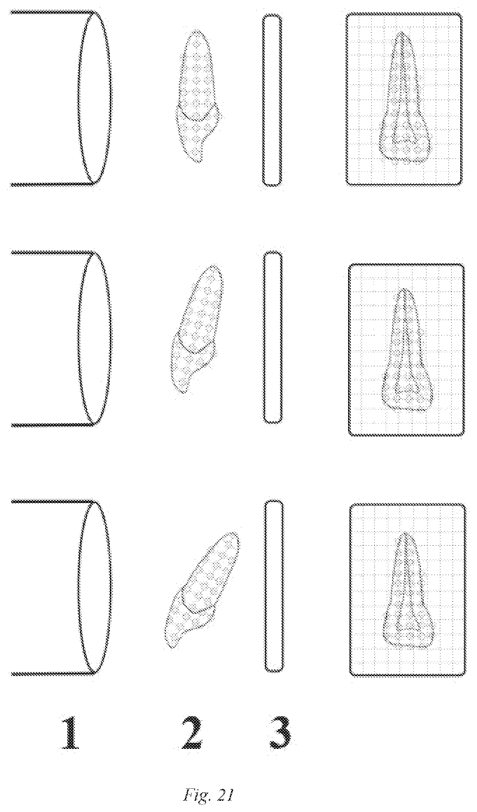

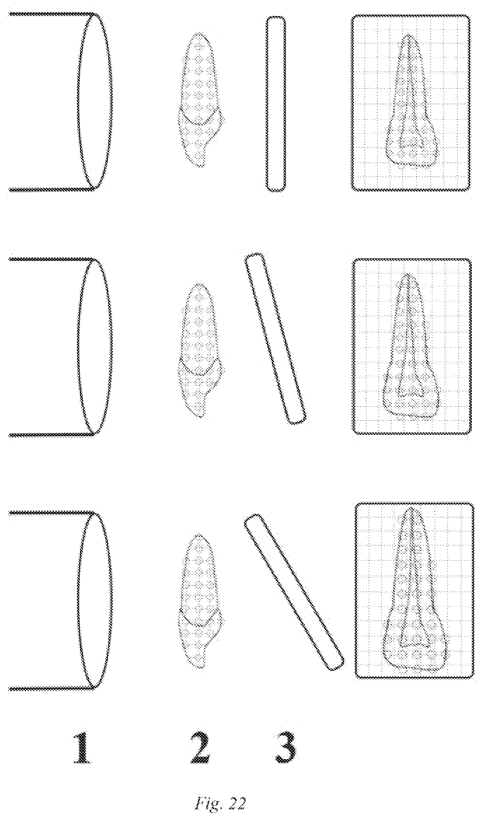

FIG. 20 schematically illustrates differences between images of a tooth taken at different angles of the light source 1 in this embodiment, with the tooth model 2 and the imaging plate 3 being maintained at the same positions. FIG. 21 schematically illustrates differences between images of a tooth taken at different angles of the tooth model 2 in this embodiment, with the light source 1 and the imaging plate 3 being maintained at the same positions. FIG. 22 schematically illustrates differences between images of the tooth taken at different angles of the imaging plate 3 in this embodiment, with the light source 1 and the tooth model 2 being maintained at the same positions. In other similar embodiments, it is also possible to take images at different parameter values of any combination of the light source 1, the tooth model 2 and the imaging plate 3 and observe differences between these images. This can help a user accumulate experience. The three components, i.e., the light source 1, the tooth model 2 and the imaging plate 3, may be dynamically varied as desired, and patterns of variation of images of nodes in an area of interest on the imaging plate 3 can be observed. Variations of tooth images with relative orientations of the light source 1, the tooth model 2 and the imaging plate 3 can be observed by naked eye. The tooth may be meshed into individual, but related small areas of interest. Observing these small areas can facilitate the establishment of various imaging techniques, training on these techniques and cultivation of thinking power about 2D-to-3D and 3D-to-2D conversion.

In the teaching apparatus of this embodiment, the light source 1 capable of producing visible light, the tooth model 2, which mimics a tooth and is meshed with the structured marking nodes, and the imaging plate 3, on which an image of the tooth model can be formed, enables intuitive reproduction of interaction among an X-ray tube, a tooth and an imaging plate. Through observing images of the structured tooth in the 3D space, the principles of the bisecting-angle and paralleling projection techniques, as well as many personalized projection techniques, can be intuitively demonstrated. This can additionally enhance the operator's perception of the essence of imaging technology, facilitating scientific research and education and offering significant benefits to periapical radiography.

In this embodiment, in order to accurately reproduce an X-ray-based dental imaging scenario, visible light is used to simulate X-ray projection for intraoral periapical radiography, and the three important components that are based on structured modeling, i.e., the light source, the tooth model and the imaging plate, are provided to model and simulate the three elements essential for X-ray radiography: a light source, an object and an image.

Through simulating X-ray projection involved in intraoral periapical radiography, this teaching apparatus can reproduce the influence of variation of the three elements essential for X-ray radiography on imaging quality. For example, changes in imaging quality resulting from varying any combination of the direction of a light beam emitted from the light source, the position of the tooth and the position of the imaging plate can be demonstrated in real time.

At first, the three essential elements are established innovatively.

### 1. Light Source

In this teaching methodology, a light source capable of producing a parallel visible light beam is used to replace and simulate the emission and projection of an invisible X-ray beam from an X-ray tube in equipment for intraoral periapical radiography.

The light source can be adjusted to produce a parallel visible light beam at a desired angle in the same manner as an X-ray tube is adjusted, for example, moved upwards, downwards, forwards or backwards, or tilted, according to the spatial position of a tooth to be imaged during periapical projection.

The use of visible light allows learning, by naked-eye observation, the influence of moving a light beam on an image of an object formed thereby. In addition, human eyes are more sensitive to visible light, and such light is free of the hazards of X-ray radiation, such as penetration and ionization.

Visible light projection can reproduce the projection of invisible, penetrating X-ray radiation. Moreover, a light beam can be emitted from the light source in a direction that can be flexibly adjusted, imparting adjustability and measurability to the projection direction and angle.

### 2. Tooth Model

It is well known that X-rays can pass through a tooth due to its nature as a kind of radiation. Due to different densities of the internal tissues of the tooth, an image of the tooth formed on an image receptor shows features allowing distinguishing the tissues from one another. Differing from the tooth, which is a 3D object, the image is two-dimensional, and a lot of information about the tooth will be lost during this 3D-to-2D conversion process due to many factors including density differences and penetration of X-rays.

In this embodiment, the tooth model mimics a tooth so as to possess various properties of the tooth. It features nodes (intersections) defining a mesh or matrix and can be personalized in various aspects, from surface to internal structure, to shape, to color, and to variability, such as the number of roots, furcation dimensions, form and number of root canals, etc. The tooth model can be titled and rotated in space, allowing derivation of a spatial transformation from an adjacent tooth to an embedded tooth. A parallel visible light beam may pass through the meshed tooth model, projecting an image of each node in the 3D space onto the imaging plate. Based on image positions of the marking nodes, imaging patterns of the internal structure, surface root canals and the like of the mimicked tooth can be derived.

The meshed tooth model of this embodiment can model a tooth with the heterogeneous marking nodes, which are arranged into a transmissive mesh or matrix resembling the shape of the tooth. The mesh nodes represent feature points on the surface of and within the tooth and can be identified by naked eye. The meshed tooth model can be visually seen through and may include nodes that mark tooth surface features such as the mesial, distal and central marginal ridges, cusps, root furcation and apical area. Additional nodes may be included to mark paths of canals within the tooth root. Each canal may be marked with multiple nodes of the same type to indicate the continuity of the path.

The surface modes may be connected with fine lines so as to define the shape of the tooth. The internal mesh modes may also be connected with fine lines so as to define the paths of root canals. Given the fact that the form of a tooth root is variable, corresponding nodes may be flexibly arranged to model, for example, curvatures, furcation dimensions, the number and paths of canals, etc.

The mesh nodes in the tooth model may be capable of special marking. In some embodiments, for example, triangular nodes may be arranged on the side closer to the light source (mesial nodes), and circular nodes may be arranged on the side farther away from the light source (distal nodes). The mesh nodes in the tooth model may also be marked with different colors. In this case, the colors may be helpful in determining positions during imaging. In some embodiments, for example, paths of root canals may be marked red nodes and lines, the crown with blue nodes, the mesial root with yellow nodes, the distal root with purple nodes, and the palatal root with black nodes. In this way, determinations may be made during imaging not only by the shapes of the nodes but also by their colors. The mesh nodes in the tooth model may also be marked with different thicknesses of lines that connect them. The marking nodes may be equidistantly spaced to facilitate determining deformations based on distance variations of the nodes in a captured image.

Either one or more such tooth models may be included. A tooth portion of particular interest (e.g., the pulp cavity, the root, or a particular external or internal site) may be marked with special marking nodes. Marking nodes (of different forms) selected from the tooth model may be even attached to a meshed tooth surface, or a pulp component may be mounted and desirable locating nodes may be attached thereto. A plurality of tooth models may be used to simulate changes in a positional relationship of adjacent teeth. Each tooth model may assume various spatial configurations. For example, it may be tilted or rotated in space. For example, long axes of adjacent teeth may be non-parallel.

The spatial position of each node can be identified by human eye. Relationships among the orientation of the visible light source, the positions of nodes in the tooth model and dynamic changes of an image of the model tooth on the imaging plate can be determined. The tooth model allows locating in the 3D space based on the nodes taken as points, lines connecting such points, and planes defined by three points or a combination of point(s) and/or lines(s). An observer can selectively observe a node of interest and positional changes of a projected image of the node formed by visible light on the imaging plate.

### 3. Graduated Imaging Plate

The graduated imaging plate facilitates measuring of nodes in an image of the tooth model formed thereon, determining a degree of matching between actual distances between the nodes and distances between their projections, observing image variation in response to projection direction, angle and distance variation, and cultivating thinking power about conversion from the marking nodes in the 3D space to their projections on a 2D plane and reverse conversion from an image of the tooth model on a 2D plane to its configuration in the 3D space.

The imaging plate can present a complete 2D image of the tooth model (i.e., an image of the meshed simulating tooth model formed by a projected visible light beam). The image may form as a result of projection of visible light onto the marking nodes, connections and surfaces. Once spatial positions of the marking nodes and connections have been clearly defined, changes in distances between and shapes of them can be identified on the imaging plate.

The imaging plate may be displaced, tilted or rotated in space, or moved to close vicinity of or away from the tooth model. Visible graduations are provided on the imaging plate as markers for indicating geometric variation of a projected image. For example, a graduated X-Y scale in the form of a cross may be provided on the imaging plate, which allows a length of the tooth model, as well as whether a known distance between some marking nodes in the tooth mode becomes longer or shorter in the image, to be determined from an image thereof formed on the graduated plate.

Image variation of the same tooth model in response to adjusting different variables can be observed. Regularities can be identified from interaction of the three components (i.e., the visible light source, tooth model and imaging plate). As the visible light source, the tooth model and an image on the imaging plate can be also directly observed by an operator, facilitating training on thinking power about conversion from the 3D tooth model to an 2D image and about conversion from an 2D image to the 3D tooth model. This can help develop a trainee's spatial imagination ability.

All feature positions of a tooth (flexible node-based marking) can be clearly indicated by nodes in the simulating tooth model (in the node-based marking method, the nodes can provide indications by shape or even color). Moreover, through illumination by a visible light beam, each node can be definitely located on the imaging plate in real time. In order to achieve definite locating, any of multiple variables may be adjusted and the resulting changes in an image of the nodes can be observed. This allows identifying patterns of variation of points, lines, planes and the like in the tooth model with the variables.

Preferred specific embodiments of the present invention have been described in detail above. It is to be understood that those of ordinary skill in the art can make various modifications and changes based on the concept of the present invention without exerting any creative effort. Accordingly, all variant embodiments that can be obtained by those skilled in the art by logical analysis, inference or limited experimentation in accordance with the concept of the present invention on the basis of the prior art are intended to fall within the protection scope as defined by the claims.

1. A dental imaging teaching apparatus comprising a tooth model, wherein the tooth model is at least partially transparent to visible light.

2. The dental imaging teaching apparatus of claim 1, wherein the tooth model comprises a plurality of nodes and connections, the plurality of nodes connected by the connections, thus forming the tooth model in the form of a mesh.

3. The dental imaging teaching apparatus of claim 2, wherein the connections are configured to be deformable to change relative distances and positions of the plurality of nodes.

4. The dental imaging teaching apparatus of claim 3, wherein the tooth model comprises a first set of nodes having a cross-section of a first shape, the first set of nodes configured to indicate a mesial area of the tooth model.

5. The dental imaging teaching apparatus of claim 3, wherein the tooth model comprises a second set of nodes having a cross-section of a second shape, the second set of nodes configured to indicate a middle area of the tooth model.

6. The dental imaging teaching apparatus of claim 3, wherein the tooth model comprises a third set of nodes having a cross-section of a third shape, the third set of nodes configured to indicate a distal area of the tooth model.

7. The dental imaging teaching apparatus of claim 3, wherein the tooth model comprises a fourth set of nodes having a cross-section of a fourth shape, the fourth set of nodes configured to indicate a pulp of the tooth model.

8. The dental imaging teaching apparatus of claim 3, wherein the tooth model comprises a fifth set of nodes having a cross-section of a fifth shape, the fifth set of nodes configured to indicate a coronal plane of the tooth model.

9. The dental imaging teaching apparatus of claim 3, wherein the tooth model comprises a sixth set of nodes having a cross-section of a sixth shape, the sixth set of nodes configured to indicate a transverse plane of the tooth model.

10. The dental imaging teaching apparatus of claim 3, wherein the tooth model comprises a seventh set of nodes having a cross-section of a seventh shape, the seventh set of nodes configured to indicate a sagittal plane of the tooth model.

11. The dental imaging teaching apparatus of claim 3, wherein, in the tooth model, the other space than a space occupied by the nodes and the connections is filled with a material at least partially transparent to visible light.

12. The dental imaging teaching apparatus of claim 3, wherein, in the tooth model, the nodes and the connections are made of materials at least partially transparent to visible light.

13. The dental imaging teaching apparatus of claim 1, further comprising a light source, the light source comprising a light source body, a light source support and a light source base, the light source body connected to the light source base by the light source support.

14. The dental imaging teaching apparatus of claim 13, wherein the light source body is configured to emit visible light.

15. The dental imaging teaching apparatus of claim 13, wherein a first movable device is provided at a junction of the light source support and the light source base, the first movable device used to adjust the light source in height and angle.

16. The dental imaging teaching apparatus of claim 1, wherein the tooth model comprises a tooth model body, a tooth model support and a tooth model base, the tooth model body connected to the tooth model base by the tooth model support.

17. The dental imaging teaching apparatus of claim 16, wherein a second movable device is provided at a junction of the tooth model support and the tooth model base, the second movable device used to adjust the tooth model in height and angle.

18. The dental imaging teaching apparatus of claim 1, further comprising an imaging plate, the imaging plate comprising an imaging plate body, an imaging plate support and an imaging plate base, the imaging plate body connected to the imaging plate base by the imaging plate support.

19. The dental imaging teaching apparatus of claim 18, wherein a third movable device is provided at a junction of the imaging plate support and the imaging plate base, the third movable device used to adjust the imaging plate in height and angle.

20. The dental imaging teaching apparatus of claim 18, wherein the imaging plate body is provided with graduations in the form of a grid.
